(19) United States
(12) Patent Application Publication
(10) Pub. No.: US 2026/0108463 A1
(43) Pub. Date: Apr. 23, 2026

(54) NANOEMULSION FORMULATION WITH IMPROVED TACROLIMUS STABILITY AND SKIN PENETRATION

(71) Applicant: BIOFRONTERA BIOSCIENCE GMBH, Leverkusen (DE)

(72) Inventors: Montserrat FOGUET, Leverkusen (DE); Mirella GWAREK, Leverkusen (DE); Lars MÖLLMANN, Leverkusen (DE)

(21) Appl. No.: 19/334,377

(22) Filed: Sep. 19, 2025

Related U.S. Application Data
(63) Continuation of application No. PCT/EP2024/059365, filed on Apr. 5, 2024.

(30) Foreign Application Priority Data
Apr. 6, 2023 (EP) 23167169.4
Apr. 6, 2023 (WO) PCT/EP2023/059292
Dec. 6, 2023 (EP) 23214699.3

(51) Int. Cl. A61K 9/107; A61K 9/00; A61K 9/06; A61K 31/436; A61K 47/10; A61K 47/14; A61K 47/24; A61K 47/26

(52) U.S. Cl. CPC A61K 9/1075; A61K 9/0014; A61K 9/06; A61K 31/436; A61K 47/10; A61K 47/14; A61K 47/24; A61K 47/26

(57) ABSTRACT

The present invention relates to a composition comprising an oil in water nanoemulsion and a highly lipophilic macrolide lactone as active agent, such as tacrolimus, dissolved in the nanoemulsion. In this formulation, tacrolimus can be fully dissolved, rather than suspended, and shows an improved stability in terms of active substance content, pH, particle size and particle size homogeneity.

The present invention relates to a composition comprising an oil in water nanoemulsion and a highly lipophilic macrolide lactone as an active agent, such as tacrolimus, dissolved in the nanoemulsion. In this formulation, tacrolimus can be fully dissolved, rather than suspended, and shows an improved stability in terms of active substance content, pH, particle size and particle size homogeneity.

### CROSS-REFERENCE TO RELATED APPLICATIONS

This Patent Application is a continuation of International Patent Application No. PCT/EP2024/059365 filed on Apr. 5, 2024, which claims priority from European Patent Application No. 23214699.3, filed Dec. 6, 2023, European Patent Application No. 23167169.4, filed on Apr. 6, 2023, and International Patent Application No. PCT/EP2023/059292, filed on Apr. 6, 2023, the entire disclosures of which are incorporated herein by reference.

### BACKGROUND OF THE INVENTION

Dispersions are colloidal systems, which include micelles, liposomes, virosomes, emulsions and micro-, nanoemulsions, suspensions and polymer solutions. Emulsions or micro emulsions can be oil in water, water in oil or middle phased dispersions, often containing surfactants as emulsifiers. Nanoemulsions are a subgroup of the emulsions that contain very fine oil in water dispersions. Nanoemulsions are highly homogeneous, transparent, and slightly opalescent. The dispersed droplets (liquid) or vesicles in such emulsions are composed of a lipid core surrounded by at least one surfactant or emulsifier monolayer. Nanoemulsions are characterized by a mean particle or vesicle size of less than 200 nm, often less than 100 nm and a narrow monodisperse particle or vesicle size distribution.

Although nanoemulsions are generally thermodynamically more stable than conventional emulsions, they are often not stable in stress situations such as high temperature or freezing conditions. Nanoemulsions can be in a metastable state and the structure depends often on the manufacturing process, making them complicated to formulate to a pharmaceutical composition with a long shelf-life under different storage conditions. If destabilized they can become heterogeneous, milky and/or exhibit phase separation. On the other side, nanoemulsions can provide useful applications in skin care in that they may exhibit good textural and sensual properties due to the very fine droplet or globule size.

Nanoemulsions are often manufactured by mechanical fragmentation of an oily phase in an aqueous phase in the presence of a surfactant. The very small size of the oily globules is often obtained by virtue of at least one pass through a high-pressure homogenizer or a sonicator.

Tacrolimus (also termed herein “TC”) is a macrolide lactone molecule harvested from the soil bacterium Streptomyces tsukubaensis. In pharmaceutical medicine, it is described as a calcineurin inhibitor with immunosuppressant capacity. It is applied topically to treat immune system mediated skin conditions such as atopic dermatitis or psoriasis. TC is a molecule with a molecular weight of 804.03 g/mol and very lipophilic properties (logP>3), i.e. by six orders of magnitude more lipophilic than ALA. Due to its very lipophilic nature, TC has been formulated in mixtures of mineral oil, paraffin, propylene carbonate, white petrolatum and white wax. TC in aqueous compositions has been formulated as suspensions and it has previously been found to be weakly stable in aqueous formulations (approx. 3 months at room temperature or up to 9 months at 5° C.).

Liquid formulations of TC in predominately water-based systems (such as nanoemulsions) have not been commercialized as finished drug product by pharmaceutical companies so far, likely hampered by the challenges of solubilizing and stabilizing it in such formulations. With the pharmaceutical use of a topical formulation of TC, two additional challenges exist. The first being the high lipophilicity, which may hinder its release from a fat-based formulation into the skin. The other being its weak ability to distribute in the skin's watery compartments (such as living cells).

### SUMMARY OF THE INVENTION

A first aspect of the invention relates to a formulation comprising (a) a nanoemulsion comprising: (i) at least one aqueous component; (ii) a carrier component comprising: (1) at least one lipophilic component, (2) at least one surfactant, and (3) at least one alcohol; and (b) an active agent, wherein the active agent is a highly lipophilic macrolide lactone.

Another aspect of the invention relates to the formulation of the first aspect for use in medicine.

Another aspect of the invention relates to the formulation of the first aspect for use a method of treatment or prevention of a dermatological, ophthalmic or autoimmune disease or condition, or for the prevention of organ rejection after transplantation.

### DETAILED DESCRIPTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention, which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Kölbl, H. eds. (1995), Helvetica Chimica Acta, CH-4010 Basel, Switzerland).

Several documents are cited throughout the text of this specification. Each of the documents cited herein (including all patents, patent applications, scientific publications, manufacturers' specifications, instructions etc.), whether supra or infra, is hereby incorporated by reference in its entirety.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments, which combine the explicitly described embodiments with any number of the disclosed and/or preferred elements. Furthermore, any permutations and combinations of all described elements in this application should be considered disclosed by the description of the present application unless the context indicates otherwise.

Throughout this specification and the claims which follow, unless the context requires otherwise, the word “comprise”, and variations such as “comprises” and “comprising”, are to be understood to imply the inclusion of a stated integer or step or group of integers or steps but not the exclusion of any other integer or step or group of integer or step. As used in this specification and the appended claims, the singular forms “a”, “an”, and “the” include plural referents, unless the content clearly dictates otherwise.

Formulating a pharmaceutical composition is a highly complex process, which needs to take account of different aspects, such as pH, solubility, polymorphism, applicability, and overall stability of the pharmaceutical composition. Additionally, one must consider the benefits and constraints of the active pharmaceutical ingredient (API), excipients, the interaction of all components and the manufacturing process. In oil in water nanoemulsion formulations, there are two different phases, which may need to be stabilized and compliant with patients: one is the hydrophobic carrier component, which is usually the carrier and needs to stabilize and release the API and the other one the aqueous component. All these aspects lead to complex formulations with a large number of ingredients. One rather attractive property oil in water nanoemulsion formulations is its ability to enhance penetration of active ingredients.

The prior art describes aqueous pharmaceutical formulations of TC as suspensions with short stability (approx. 3 months at room temperature or up to 9 months at 5° C.).

The prior art fails to teach a pharmaceutical aqueous composition able to solubilize tacrolimus, a highly lipophilic active agent. Furthermore, prior art fails to teach aqueous formulations (in solution or suspension) for tacrolimus with an extended stability over 24 months.

As described above, nanoemulsions tend to coalesce under certain circumstances, such as exposure to extreme temperature differences, leading to bigger droplet sizes and harm the nanoemulsion quality.

These aspects make it clear that design and formulation of a composition with a high penetration and improved impurity profile is highly desirable.

The terms “active agent” and “active ingredient” are used interchangeably herein. As used herein, “active agent”, includes an active pharmaceutical agent (herein also termed “pharmaceutical active agent” or “active pharmaceutical ingredient”, “API”) and an active cosmetic agent (herein also termed “cosmetic active agent”). As used herein, an active pharmaceutical agent is defined as the chemical, biological, mineral or any other entity or component responsible for the therapeutic (pharmacological, physiological, physical, etc.) effects in a product. As used herein, an active cosmetic agent is defined as the chemical, biological, mineral or any other entity or component responsible for the cosmetic effects in a product. The active agent may be a plant extract. The active agent may be present as a pharmaceutically acceptable salt. The active agent may be present as a cosmetically acceptable salt.

The formulations of the invention comprise two phases:
(I) an aqueous phase or aqueous component,
(II) a lipid phase or carrier component.

A first aspect of the invention relates to a formulation comprising
(a) a nanoemulsion comprising:
   (i) at least one aqueous component;
   (ii) a carrier component comprising:
      (1) at least one lipophilic component,
      (2) at least one surfactant, and
      (3) at least one alcohol; and
(b) an active agent, wherein the active agent is a highly lipophilic macrolide lactone.

In the context of the present specification, the term “highly lipophilic” refers to a compound having a logP value that is at least 3, wherein P is the octanol-water partition-coefficient.

Preferably, the active agent has a logP value of 3 or higher, such as 3.0 to 7.0 or 3.0 to 5.0.

In the context of the present specification, the term “macrolide lactone” refers to a compound comprising or consisting of a macrocyclic lactone ring. One or more deoxy sugars can be attached to the lactone ring. It is preferred that the macrocyclic lactone ring comprises at least one cyclic half acetal, one 1,2-dicarbonyl substructure as well as one piperidine substructure. Preferably, the macrocyclic lactone ring comprises two methoxy ligands at position 14 and 16. Preferably, the macrolide lactone is not halogenated.

Preferably, the active agent has immunosuppressant capacity.

It is particularly preferred that the active agent is Tacrolimus, Pimecrolimus, Everolimus or Sirolimus, preferably Tacrolimus, a derivative, an isomeric form, a tautomeric form, a precursor, a metabolite, hydrate, and/or a pharmaceutically acceptable salt thereof.

Tacrolimus, also termed “TC” herein, is identified by CAS number 104987-11-3 and has the following chemical formula:

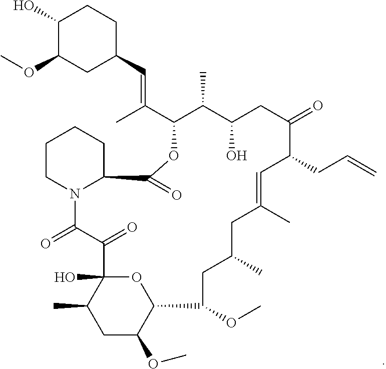 [SMILES: C=CC[C@@H]1/C=C(\C)C[C@H](C)C[C@H](OC)[C@H]2O[C@@](O)(C(=O)C(=O)N3CCCC[C@H]3C(=O)O[C@H](/C(C)=C/[C@@H]3CC[C@@H](O)[C@H](OC)C3)[C@H](C)[C@@H](O)CC1=O)[C@H](C)C[C@@H]2OC]

Tacrolimus is a macrolide lactone molecule harvested from the soil bacterium Streptomyces tsukubaensis. In pharmaceutical medicine, it is described as a calcineurin inhibitor with immunosuppressant capacity. It is applied topically to treat immune system mediated skin conditions such as atopic dermatitis or psoriasis. TC is a molecule with a molecular weight of 804.03 g/mol and very lipophilic properties (logP>3), i.e. by six orders of magnitude more lipophilic than 5-aminolevulinic acid (ALA). Due to its very lipophilic nature, TC has been formulated in mixtures of mineral oil, paraffin, propylene carbonate, white petrolatum and white wax. TC has previously been found to be weakly stable in aqueous formulations (approx. 90 days at room temperature).

Formulations of TC in predominately water-based semisolid systems (such as emulsions) have not been put to pharmaceutical use so far, likely hampered by the challenges of solubilizing and stabilizing it in such formulations. With the pharmaceutical use of TC, two challenges exist. The first being the high lipophilicity, which may hinder its release from a fat-based formulation into the skin. The other being its weak ability to distribute in the skin's watery compartments (such as living cells).

A precursor of TC is e.g. Pre-Tacrolimus, which has the following chemical formula:

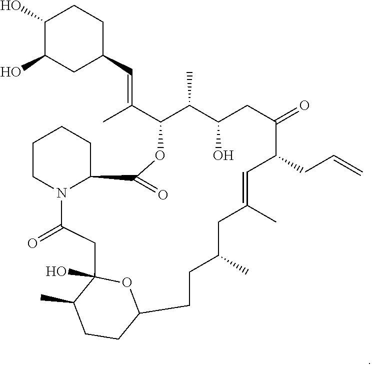 [SMILES: C=CC[C@@H]1/C=C(\C)C[C@H](C)CCC2CC[C@@H](C)[C@](O)(CC(=O)N3CCCC[C@H]3C(=O)O[C@H](/C(C)=C/[C@@H]3CC[C@@H](O)[C@H](O)C3)[C@H](C)[C@@H](O)CC1=O)O2]

Sirolimus is identified by CAS number 53123-88-9 and has the following chemical formula:

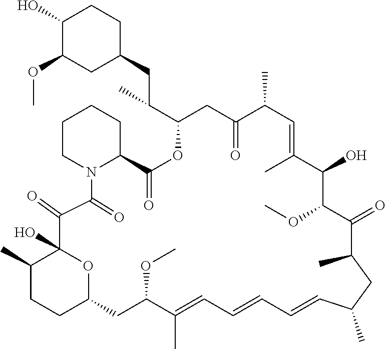 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)C(=O)[C@H](C)C[C@H](C)/C=C/C=C/C=C/1C]

A precursor of Sirolimus is e.g. Pre-Sirolimus, which has the following chemical formula:

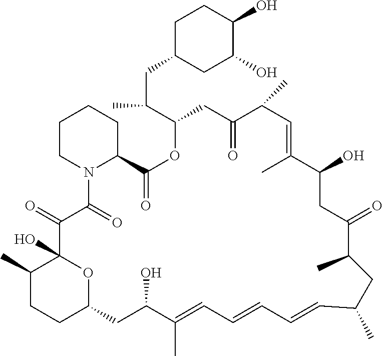 [SMILES: C/C1=C\[C@@H](C)C(=O)C[C@@H]([C@H](C)C[C@@H]2CC[C@@H](O)[C@H](O)C2)OC(=O)[C@@H]2CCCCN2C(=O)C(=O)[C@]2(O)O[C@@H](CC[C@H]2C)C[C@H](O)/C(C)=C/C=C/C=C/[C@@H](C)C[C@@H](C)C(=O)C[C@@H]1O]

Pimecrolimus is identified by CAS number 137071-32-0.

Everolimus is identified by CAS number 159351-69-6.

In preferred embodiments, the formulation is a pharmaceutical formulation.

In some embodiments, the formulation is a lotion, a spray, a foam, an emulsion, a nanoemulsion, a gel or a cream. In some embodiments, the formulation is a lotion. In the context of the present specification, a lotion is a low-viscosity topical preparation intended for application to the skin. A lotion has a lower viscosity than a cream or a gel due to its higher water content. In some embodiments, the lotion has a viscosity of ≤8 Pa s (pascal-second), ≤6 Pas, ≤5 Pas, ≤4 Pas, ≤3 Pas, ≤1.0 Pas, or ≤0.5 Pa s.

The skilled person is aware of suitable methods for determining the viscosity. Preferably, the viscosity is determined as described in the examples section.

The formulation may be for topical, ophthalmic or systemic use. In preferred embodiments, the formulation is for topical use.

In preferred embodiments of the formulations described in the present invention, the aqueous component comprises an aqueous phase or forms an aqueous phase.

In preferred embodiments of the formulations described in the present invention, the carrier component comprises or consists of nanovesicles. The carrier component can also be referred to as the lipid phase of the nanoemulsion. Preferably, the active agent is dissolved in the lipid phase of the nanovesicles. In other words, the active agent is dissolved in the lipid phase of the nanoemulsion.

The active agent may be present as a salt, hydrate or derivative.

The small size of the nanovesicles and their high homogeneity confers on them advantageous properties which distinguish them from conventional emulsions: The nanoemulsions and formulations comprising nanoemulsions of the present invention are transparent. Further, the nanoemulsion and formulations comprising nanoemulsions of the present invention can carry active agents such as tacrolimus more efficiently and, thus, become increasingly important in the field of medicine and pharmacy.

“Aging” as used herein, refers to alteration, disintegration and/or degradation of the formulation, affecting chemical and physical stability during storage, in particular under stressed conditions. Such physical or chemical changes due to storage may include, but are not limited to Ostwald ripening, flocculation, coalescence and/or breaking, which may lead to a change in vesicle size or polydispersity index.”

The inventors have found that tacrolimus can surprisingly be dissolved, rather than suspended, in the aqueous formulations of the present invention containing nanoemulsion.

The inventors have furthermore found that the formulations of the present invention are surprisingly stable and resistant to aging. In particular, the formulations of the present invention are stable in terms of TC content and particle size and particle size distribution even after storage, for example for 24 months at 2-8° C.

In the context of the present specification, whenever a duration is described as “one month, two months, three months” etc., this is meant to include embodiments in which the duration is “at least one month, at least two months, at least three months” etc.

As used herein, a “nanovesicle emulsion” or a “nanoemulsion” is a dispersion of oil in water (oil-in-water dispersion, oil-in-water emulsion, O/W emulsion). The nanoemulsion can be monophasic, transparent and/or slightly opalescent. The nanoemulsions of the present invention can be colloidal systems, which include dispersed nanovesicles comprising a lipid core surrounded by at least one surfactant or emulsifier monolayers. The nanoemulsions and the formulations comprising the nanoemulsions of the present invention are characterized by a mean particle or nanovesicle size of less than 500 nm less, than 200 nm, or less than 100 nm. The nanoemulsions and the formulations comprising the nanoemulsions of the present invention has a narrow (homogeneous) nanovesicle size distribution, for example a nanovesicle size distribution characterized by a polydispersity index of less than or equal to 0.4.

As used herein, “nanovesicle”, “nano vesicle”, “lipid vesicles”, “oil droplets”, “droplets” and “oil globules” are interchangeable and refer to small oil droplets in an oil in water emulsion. A lipid vesicle of an average size (see above, e.g., below 500 nm, 200 nm, 100 nm) that is compiled of a monolayer of a surfactant and a lipid core. In the present invention, the nanovesicles can have a size of less than or equal to 500 nm, or less than or equal to 300 nm, preferably in the range of 5 nm to 200 nm, more preferably in the range of 5 nm to 100 nm.

As used herein, the term “nanoparticle” or “nano particle”, is distinguished from “nanovesicles”, and refers to solid particles, which are not described in this invention. The formulation of the present invention may be a formulation which is essentially free of nanoparticles. “Essentially free of nanoparticles” means, that the formulation comprises less than or equal to 2% by weight, or less than or equal to 1% by weight of, or does not comprise nanoparticles. Nanoparticles are mainly inorganic, solid lipids or polymeric solid particles may have a size of below 100 nm, below 200 nm, or below 500 nm. The size can be determined by the methods as described herein. For example, the formulation may be essentially free of nanoparticles with a diameter of less than 100 nm, as determined by dynamic light scattering.

As used herein, “topical use” or “topical treatment” of the formulation of the invention describes an application to a particular place on or in the body, in particular the human body. This includes, but is not limited to administration of the formulation to body surfaces such as the skin or mucous membranes. The topical use can be epicutaneous, meaning that the formulation is directly administered to the skin. In particular, the topical use is a pharmaceutical use.

As used herein, “systemic use” or “systemic treatment” of the formulation of the invention describes an application in which the active agent is distributed throughout the body via the blood or lymphatic system, for example after an injection or oral intake.

As used herein, the “stability” of a formulation comprising nanovesicles, as described herein, includes, but is not limited to the physical and chemical stability. In particular, in the present invention, a formulation is stable if the integrity of the nanovesicles is found to be stable. A measure known to the skilled person to describe integrity of the nanovesicles is the size, as for example determined by dynamic light scattering, as described herein. The nanovesicles produced according to the invention can have a size below 100 nm, preferably below 50 nm, more preferably in the range of 20 nm to 30 nm, immediately after manufacture. For example, the formulation as described herein is stable if the nanovesicles in the formulation of the present invention have a size (or diameter) of less than or equal to 500 nm or less than or equal to 300 nm, preferably in the range of 5 nm to 200 nm, more preferably in the range of 5 nm to 100 nm.

“Stability” can also refer to the absence of processes above described as aging, leading to a loss of pharmaceutical functionality or quality. The composition described in this invention is functional or pharmaceutically functional, as long as the vesicle size is less than or equal to 500 nm or less than or equal to 300 nm, preferably in the range of 5 nm to 200 nm, more preferably in the range of 5 nm to 100 nm.

Furthermore, “stability” can refer to the stable content of the active agent, in particular tacrolimus. During storage, the content of the active agent is, for example, considered stable if at least 70%, at least 80% or at least 90% of the content of the active agent is still present, when stored, for example, at stressed conditions, as described herein.

In a formulation of the invention, the content of the active agent may be
(i) at least 90% after storage for 1 months at 40° C., and/or
(ii) at least 90% after storage for 3 or 6 months at 25° C., and/or
(iii) at least 90% after storage for 12, 18 or 24 months at 2-8° C.

In the present invention, the active agent of the invention can be stable for at least one month, at least 3 months, at least 6 months, at least 9 months, at least 12 months or at least 24 months, at 2-8° C., or at about 5° C.

In the present invention, the formulation of the invention can be stable for at least one month, at least 3 months, at least 6 months, at least 9 months, at least 12 months, or at least 24 months, at 2-25° C., at 2-8° C., at 15-25° C., at 25° C. or at about 5° C.

The formulation of the present invention may have a nanovesicle size of less than or equal to 500 nm, or less than or equal to 300 nm, or less than or equal to 200 nm, preferably in the range of 5 nm to 200 nm, more preferably in the range of 5 nm to 100 nm, when stored for 3 months at 40° C.

The formulation of the present invention may have a nanovesicle size of less than or equal to 500 nm, or less than or equal to 300 nm, or less than or equal to 200 nm, preferably in the range of 5 nm to 100 nm, when stored for 24 months at 25° C.

Preferably, the active agent has a content of more than or equal to 80%, preferably more or equal to 85%, more preferably more or equal to 90% when
a) stored for one month, two months, three months, six months at 2-25° C.; or
b) stored for one month, two months, three months, six months, twelve months, eighteen months or twenty-four months at 2-8° C.

Preferably, the nanoemulsion comprises nanovesicles, wherein the nanovesicles have a size of less than or equal to 500 nm, preferably less than or equal to 200 nm, more preferably in the range of 5 nm to 100 nm when
a) stored for one month, two months, three months, six months, twelve months, eighteen months, or twenty-four months at 2-25° C.; or
b) stored for one month, two months, three months, six months, twelve months, eighteen months, twenty-four months, thirty months, or thirty-six months at 2-8° C.

Preferably, the polydispersity index of the formulation is less than or equal to 0.4 when
a) stored for one month, two months, three months, six months, twelve months, eighteen months, or twenty-four months at 2-25° C.
b) stored for one month, two months, three months, six months, twelve months, eighteen months, twenty-four months, thirty months, thirty-six months at 2-8° C.

The size or diameter of the nanovesicles as described herein can be expressed as the Z-average (also termed “z-average”). The size distribution of the nanovesicles can be characterized by the polydispersity index. These parameters are well known to the skilled person, and are widely used in the art to characterize particle or vesicles in emulsions, suspensions and/or polymeric solutions.

In the present invention, the size of the nanovesicles (e.g., z-average in nm) and/or the heterogeneity of nanovesicle formulations (characterized by the polydispersity index) can be determined by dynamic light scattering (also referred as Photon Correlation Spectroscopy (PCS) or Quasi-Elastic Light Scattering (QELS)). Dynamic light scattering is well known in the art and well established to determine size of nano or micro particles or vesicles in emulsions, suspensions and/or polymeric solutions with a laser.

In the formulation as described herein, the total aqueous component can present in an amount of 50% to 99% w/w, based on the total weight of the nanoemulsion (a), preferably from 70% to 95% (w/w), and more preferably from 75% to 95% (w/w), or 80% to 95%.

As used herein, “weight per weight”, “weight/weight” or “w/w” means the weight concentration or mass concentration of a component in a formulation described herein. The weight or mass of a component is expressed as a percentage of a reference formulation. For example, the weight or mass of a component can be expressed as a percentage of the total weight or mass of the formulation of the invention, or as a percentage of the total weight or mass of the nanoemulsion (a).

The aqueous component can comprise at least one pH buffering agent. Any suitable buffering agent may be used. Suitable buffering agents are known to the skilled person. For example, the at least one pH buffering agent can be selected from the group consisting of citrate, phosphate, acetate and carbonate.

The pH of the aqueous component can be in the range of 2-9. The pH of the aqueous component can also be preferably in the range of 2-6, such as 2, 3, 4, 5 or 6, more preferably in the range of 3-6, such as 3, 4, 5 or 6, or 3-5, such as 3, 4 or 5. In other embodiments, the pH of the aqueous component can also be in the range of 4-10, or 5-7, preferably about 7.4.

In instances where the formulation is a topical or oral formulation, the pH of the formulation is 2-7, preferably 2-6, more preferably 3-5. In instances where the formulation is a parenteral or ophthalmic formulation, the pH of the formulation is 4-10, or 5-7, preferably about 7.4.

In the formulation as described herein, the at least one lipophilic component can be selected from triglycerides and mixtures thereof.

Preferably, the at least one lipophilic component is a lipid, a synthetic oil, a vegetable oil and/or an animal oil. Suitable lipids according to the present invention are physiologically acceptable lipids such as ceramide, mono-, di- and triacylglycerin (triglycerides). In particular, the at least one lipophilic component is a triglyceride, preferably a triglyceride comprising a C₈₋₁₀ fatty acid, or a mixture thereof. More particular, the at least one lipophilic component is a caprylic and/or capric triglyceride and/or a mixture thereof, particularly preferably Miglyol (such as Miglyol 812, available e.g., from IOI Oleochemical) or Myritol (such as Myritol 318, available e.g., from BASF). Suitable vegetable and animal oils e.g., are sunflower oil, soybean oil, peanut oil, rape oil, fish oil and/or cetaceum.

In the formulation as described herein, the at least one lipophilic component can be present in an amount of from 0.1% to 30% (w/w) based on the total weight of the nanoemulsion (a), preferably from 0.25% to 15% (w/w), preferably from 0.25% to 10% (w/w), and more preferably from 0.5% to 8% (w/w) or 3% to 8% (w/w). Also preferred is the at least one lipophilic component being present in an amount of from 10% to 30% (w/w) based on the total weight of the nanoemulsion (a), more preferably 15-30%, or 10-20%.

In the formulation as described herein, the at least one surfactant may be any suitable surfactant known to the skilled person.

Surfactants, also referred as surface active agents or emulsifiers, are well-known in the art and include any agent linking oil and water in the composition to form an emulsion. They lower the surface tension of two liquids and are amphiphilic. In emulsions they are referred as emulsifiers and coat the droplets preventing coalescing. Emulsifiers can be described with the hydrophilic/lipophilic balance (HLB), which expresses their affinity towards water or oil. Low HLB (e.g., HLB=1) refers to lipophilic emulsifiers and high (e.g., HLB=20) to hydrophilic emulsifiers. In general, lipophilic emulsifiers are used for water-in-oil emulsions and hydrophilic emulsifiers for oil-in-water emulsions. Persons skilled in the art will identify which emulsifiers or mixtures of them are suited for the preferred vehicles and purpose of the composition. In certain emulsions, combinations of emulsifiers might be advantageous.

A suitable membrane-forming surfactant is a phospholipid, a lysophospholipid, a ceramide and/or a mixture thereof. Preferably, the phospholipid is lecithin or cephalin from soybeans or hens' eggs.

Preferably, the at least one surfactant is a phospholipid, more preferably lecithin, most preferably soy lecithin.

In the formulation as described herein, the phospholipid, in particular phosphatidylcholine, the lysophospholipid, the ceramide and/or the mixture thereof can be present in an amount of from 0.1% to 10% (w/w), based on the total weight of nanoemulsion (a), preferably from 0.15% to 5% (w/w), and more preferably from 0.2% to 3% (w/w) or from 0.2% to 4% (w/w), most preferably from 2.5% to 4% (w/w).

Preferably, the lecithin has a phosphatidylcholine content of at least 80% by weight, more preferably of at least 90% by weight, and most preferably of at least 94% by weight. The quality of the lecithin, namely its phosphatidylcholine content, plays a crucial role for the size of the vesicles of the nanoemulsion. The higher the phosphatidylcholine content of the lecithin, the smaller is the size of the vesicles of the nanoemulsion.

As O/W emulsion-forming surfactant, anionic, nonionic, cationic and/or amphoteric surfactants are suitable as well as block copolymers. Suitable anionic surfactants are soaps, alkylbenzene sulphonates, alkane sulphonates, alkylsulfates and/or alkyl ether sulfates. Suitable cationic surfactants are quaternary ammonium compounds, preferably having one or two hydrophobic groups (e.g., cetyltrimethylammonium bromide and cetyltrimethylammonium chloride) and/or salts of tong-chain primary amines. A suitable amphoteric surfactant Is N-(acylamidoalkyl) betaine, N-alkyl-β-aminopropionate, phosphate-alkyl-ammonium compounds, and/or amine-N-oxide. A suitable copolymer building block, for example, is propylene oxide. In the present invention, a nonionic surfactant is particularly preferred as O/W emulsion-forming surfactant.

In preferred embodiments, the surfactant is a polyoxyethylene-type surfactant. In the formulation as described herein, the at least one surfactant can be any a polyoxyethylene-type surfactant. A suitable nonionic surfactant can be selected from the group consisting of fatty alcohol polyglycolether, alkylphenol polyglycolether, alkylpolyglucoside, fatty acid glucamide, fatty acid polyglycolether, ethylen oxide-propylene oxide-block polymer, polyglycerol fatty acid ester, fatty acid alcanolamide and (ethoxylated) sorbitane fatty acid ester (sorbitane). A particularly preferred ethoxylated sorbitane fatty acid ester is polyoxyethylene sorbitane monooleate, most preferably Polysorbate 80.

The at least one surfactant, such as the polyoxyethylene-type surfactant, can be present in an amount of from 0.1% to 10% (w/w), based on the total weight of the nanoemulsion (a), more preferably from 0.2% to 5% (w/w), and most preferably from 1% to 5% (w/w) or 0.5% to 5% (w/w). The formulation of the invention can comprise as least one hydrophilic surfactant with an HLB of 9 to 17, more preferably 12-16, particularly polysorbate 80 to form a nanoemulsion.

The at least one surfactant can be a sugar-based surfactant. Sugar-based surfactants are a group of non-ionic surfactants using hydrophilic sugars to which hydrophobic tails are bound. One common substance of this class is n-dodecyl-β-D-maltoside, a member of the maltoside surfactants so named because the sugar unit used is maltose. An example of a pyranoside surfactant is n-octyl-β-D-thioglucopyranoside. This class uses pyranose as the sugar unit. Examples of the glycoside surfactants are octyl glucoside, decyl glucoside, and lauryl glucoside. An example of a polysugar surfactant is digitonin.

Another very important group of sugar-based surfactants are the Tween surfactants, most notable Tween 20 (also termed herein Polysorbate 20) and Tween 80 (also termed herein Polysorbate 80). These surfactants are based on a sorbitan sugar, which is why they are commonly referred to as polysorbate surfactants. Three oligo (ethylene glycol) side groups of varying lengths are bound to the sugar increasing the hydrophilicity of the head group. This structure forms the core of all Tween surfactants. They deviate in the hydrophobic tail, which is a fatty acid coupled via an ester to four oligo (ethylene glycol) tail. In Tween 20 this fatty acid is lauric acid; in Tween 80 it is oleic acid.

In some embodiments, the at least one surfactant is selected from the group consisting of a phospholipid, in particular phosphatidylcholine, a lysophospholipid, a ceramide and/or a mixture thereof. In some embodiments, the at least one surfactant is a polyoxyethylene-type surfactant. In some embodiments, the at least one surfactant is phosphatidylcholine. In some embodiments, the formulation comprises a phospholipid as surfactant and a polyoxyethylene-type surfactant. In some embodiments, the formulation comprises phosphatidylcholine as surfactant and a polyoxyethylene-type surfactant. In some embodiments, the formulation comprises phosphatidylcholine and polysorbate 80 as surfactants.

In some embodiments, the at least one surfactant is phosphatidylcholine.

In some embodiments, the formulation comprises from 0.5% to 5% (w/w), preferably from 1% to 4% (w/w), more preferably from 1.2% to 3.5% (w/w) phosphatidylcholine.

In some embodiments, the formulation comprises from 0.1% to 10% (w/w), preferably from 0.15% to 5% (w/w), more preferably from 0.25% to 4.5% (w/w) phosphatidylcholine.

In preferred embodiments, the at least one alcohol comprises at least three carbon atoms.

In the formulation as described herein, the at least one alcohol preferably independently has 3-5 (i.e. no more than 5) or 3-4 (i.e. no more than 4) carbon atoms. The at least one alcohol can be at least one monohydric alcohol. Particularly suitable alcohols having 5 carbon atoms are 1-pentanol and/or 4-methyl-2-pentanol. Suitable alcohols having 4 carbon atoms are 1-butyl alcohol, iso-butyl alcohol (2-methyl-1-propanol), tert-butyl alcohol (2-methyl-2-propanol) and/or sec-butyl alcohol (2-butanol). The alcohol is not propylene glycol.

Preferably, the at least one alcohol has 3 carbon atoms, i.e. is selected from the group consisting of 1-propanol or 2-propanol (isopropyl alcohol) and mixtures thereof. A preferred alcohol is 2-propanol.

In the formulation as described herein, the alcohol may present in an amount of from 0.1% to 10% w/w based on the total weight of the nanoemulsion (a), preferably from 0.5% to 5% (w/w), and more preferably from 1% to 2% (w/w).

The formulation as described herein may comprise a gelling agent. Any suitable gelling agent may be used. Suitable gelling agents and mixtures thereof are known to the skilled person. In the formulation as described herein, the gelling agent may be selected from the group consisting of poloxamer, xanthan, bentonite, sodium carboxymethylcellulose, hydroxymethyl cellulose, carbomer, hydroxypropyl cellulose, gellan gum, guar gum, pectin, poly(ethylene) oxide, polycarbophil, alginate, tragacanth, povidone, gelatin, and mixtures thereof.

It is preferred that the gelling agent is selected from poloxamer, xanthan and/or mixtures thereof.

It is also preferred that the gelling agent is xanthan (xanthan gum).

It is also preferred that the gelling agent is a poloxamer.

Poloxamers are nonionic triblock copolymers composed of a central hydrophobic chain of polyoxypropylene (poly(propylene oxide)) flanked by two hydrophilic chains of polyoxyethylene (poly(ethylene oxide)). Commercially available are Poloxamer 407 and Poloxamer 188. Poloxamer 407 can have an average molecular weight of about 12600 Dalton. Poloxamer 188 can have an average molecular weight of about 8400 Dalton. In the formulation as described herein, a preferred poloxamer is Poloxamer 407.

In the formulation as described herein, the gelling agent may be present in an amount of from 0.1% to 10% (w/w), based on the total weight of the formulation, preferably from 0.25% to 5% (w/w), and more preferably from 0.5% to 4% (w/w) or 1% to 4% (w/w).

The formulation as described herein may comprise one or more preservatives. Any suitable preservative or a mixture thereof may be used. Suitable preservatives are known to the skilled person. The preservative can be selected from benzoate, tocopherol or derivatives and any mixtures thereof, citric acid, EDTA, potassium sorbate, vitamin C and/or derivatives and any mixtures thereof, wherein the preservative is preferably sodium benzoate. Suitable aqueous mixtures of sodium benzoate and potassium sorbate are commercially available, for example Euxyl™ K 712 preservative (Ashland). These components can be part of the aqueous component and/or of the nanovesicles.

The preservative can be present in the formulation as described herein in an amount of from 0.01% to 10% (w/w), 0.01% to 7% (w/w), 0.01% to 5% (w/w), 0.01% to 3% (w/w) based on the total weight of the formulation, preferably from 0.2% to 2% (w/w) or 0.1-2% (w/w), and more preferably from 0.2% to 1.5% (w/w).

The formulation of the present invention may be a gel formulation. As used herein, a “gel” is a two-phase elastic colloidal material, consisting of a dispersed liquid incorporated in the solid phase often constituted by a gelling agent. Suitable gelling agents, such as xanthan, are described herein.

The formulation of the present invention may be provided in a container or dispenser. Suitable dispensers and containers are known to the skilled person. For example, the dispenser may be a squeeze tube, comprising the formulation as described herein. The squeeze tube may contain the gel formulation as described herein. The dispenser may also be a metered dose dispenser or a foam dispenser or spray dispenser.

The container or dispenser may comprise a propellant, wherein the propellant is provided to pressurize the container or dispenser. Any suitable propellant may be used. Suitable propellants and mixtures thereof are known to the skilled person. Preferably, the propellant is selected from propane, isobutane, n-butane and mixtures thereof.

In some embodiments, the formulation comprises a propellant and is comprised in a pressurized container.

Surprisingly, the formulation of the present invention comprised in a pressurized container has a high foaming capacity and forms a stable foam (long collapse time) when released from the pressurized container, even in the absence of a fatty alcohol or another foam adjuvant.

Surprisingly, the formulation of the present invention comprised in a pressurized container is highly stable with regard to nanovesicle size, even in the absence of a gelling agent or in the presence of only low concentrations of a gelling agent.

Surprisingly, the formulation of the present invention comprised in a pressurized container is highly stable with regard to the concentration of the active agent, even in the absence of a gelling agent or in the presence of only low concentrations of a gelling agent.

The formulation of the present invention comprised in a pressurized container is surprisingly resistant to aging at stressed conditions in respect to API content and nanoemulsion vesicle size, compared to a formulation. In the context of the present specification, the term “petrolatum” relates to a semi-solid mixture of hydrocarbons derived from the distillation of petroleum. The hydrocarbons that make up petrolatum mainly comprise at least 25 carbon atoms. The CAS number of petrolatum is 8009-03-8. Preferably, the formulation of the first aspect of the invention comprises essentially no petrolatum.

The formulation of the present invention may be prepared as a foamable formulation.

The formulation of the present invention may be prepared as a pressurized formulation, wherein a propellant is provided to pressurize the formulation. Any propellant as described herein can be used.

The formulation of the present invention may be prepared as a pressurized, foamable formulation, wherein a propellant is provided to pressurize the formulation. Any propellant as described herein can be used.

The formulation of the invention may be provided in a foam dispenser, as described herein. The foam dispenser comprises a container, wherein said container comprises the formulation as described herein, and a propellant. The propellant is provided to pressurize the foam dispenser. Any suitable propellant may be used, as described herein. A foam-generating device is mounted on the container. In particular, the formulation is prepared as a foamable formulation. Said foam-generating device can comprise a valve for releasing and dosing of the formulation, and a push button for actuating the valve. Upon actuating the push button, the formulation can be released and can form a foam. Suitable dispensers are known to the skilled person.

The formulation of the invention may be provided in a spray dispenser, as described herein. The spray dispenser comprises a container, wherein said container comprises the formulation as described herein, and a propellant. The propellant is provided to pressurize the spray dispenser. Any suitable propellant may be used, as described herein. A spray-generating device is mounted on the container. In particular, the formulation is prepared as a sprayable formulation.

The invention also provides a foam, comprising the formulation of the present invention, as described herein.

In preferred embodiments, the active agent in the formulation of the invention is a highly lipophilic active agent, such as tacrolimus.

The active agent may be present in an amount of from 0.001% to 25%, 20%, 15%, 10% or 5% w/w, based on the total weight of the formulation. In particular, the active agent may be present in an amount of from 0.001% to 10% w/w, based on the total weight of the formulation, 0.005% to 5%, or 0.01% to 0.5% w/w, based on the total weight of the formulation.

In some embodiments, the formulation is a topical formulation and the active agent is present in an amount of from 0.001% to 0.5% w/w, preferably 0.005% to 0.2% w/w, more preferably 0.01% to 0.1% w/w, based on the total weight of the formulation.

In some embodiments, the formulation is a systemic formulation and the active agent is present in an amount of from 0.005% to 25% w/w, preferably 0.01% to 10% w/w, more preferably 0.01% to 5% w/w, even more preferably 0.01% to 2% w/w, based on the total weight of the formulation. The systemic formulation may be a formulation for injection or an oral formulation. In some embodiments, the systemic formulation is diluted with a pharmaceutically acceptable buffer prior to injection, resulting in a final concentration of 0.01% to 1% w/w based on the total weight of the formulation.

In some embodiments, the formulation is an ophthalmic formulation, in particular eyes drops, and the active agent is present in an amount of from 0.001% to 0.5% w/w, preferably 0.005% to 0.2% w/w, more preferably 0.01% to 0.1% w/w, based on the total weight of the formulation.

In preferred embodiments, the formulation of the invention comprises essentially no fatty alcohol.

In the context of the present specification, the expression “comprises essentially no” “is essentially free of” specifies that the formulation is either free of a compound or comprises less than 0.5% (w/w), less than 0.4% (w/w), less than 0.3% (w/w), less than 0.2% (w/w), less than 0.1% (w/w), less than 0.08% (w/w), less than 0.07% (w/w), less than 0.06% (w/w), less than 0.05% (w/w), less than 0.04% (w/w), less than 0.03% (w/w), less than 0.02% (w/w), or less than 0.01% (w/w) of a compound based on the total weight of the formulation.

Fatty alcohols have been described to work as foam adjuvants. They are used in prior art formulations, in particular in foamable formulations.

Preferably, the formulation comprises essentially no fatty alcohol and essentially no fatty acid.

In the context of the present specification, the term “fatty alcohol” relates to alcohols having at least 6 carbon atoms, usually 6-28 carbon atoms. Fatty alcohols can be saturated or unsaturated and unbranched or branched. Fatty alcohols are usually straight-chain primary alcohols. The term “fatty alcohol” as used herein relates to fatty alcohols in their standalone form and does not include esters comprising fatty alcohols.

In the context of the present specification, the term “fatty acids” relates to carboxylic acids with an aliphatic chain of at least 6 carbon atoms, usually 6-28 carbon atoms. Fatty acids can be saturated or unsaturated and unbranched or branched. Most naturally occurring fatty acids have an unbranched chain of carbon atoms. The term “fatty acid” as used herein relates to fatty acids in their standalone form and does not include esters comprising fatty acids.

In preferred embodiments, the formulation is either free of a fatty alcohol (in its standalone form, or in other words as isolated molecule) or comprises less than 0.5% (w/w), less than 0.4% (w/w), less than 0.3% (w/w), less than 0.2% (w/w), less than 0.1% (w/w), less than 0.08% (w/w), less than 0.07% (w/w), less than 0.06% (w/w), less than 0.05% (w/w), less than 0.04% (w/w), less than 0.03% (w/w), less than 0.02% (w/w), or less than 0.01% (w/w) of a fatty alcohol (in its standalone form, or in other words as isolated molecule) based on the total weight of the formulation.

In particular, in preferred embodiments, the formulation is either free of a fatty acid and a fatty alcohol (in their standalone form, or in other words as isolated molecules) or comprises less than 0.5% (w/w), less than 0.4% (w/w), less than 0.3% (w/w), less than 0.2% (w/w), less than 0.1% (w/w), less than 0.08% (w/w), less than 0.07% (w/w), less than 0.06% (w/w), less than 0.05% (w/w), less than 0.04% (w/w), less than 0.03% (w/w), less than 0.02% (w/w), or less than 0.01% (w/w) of a fatty acid and a fatty alcohol (in their standalone form, or in other words as isolated molecules) based on the total weight of the formulation.

More preferably, the formulation comprises essentially no foam adjuvant. In particular, the formulation can be free of foam adjuvant or comprises less than 0.5% (w/w), less than 0.4% (w/w), less than 0.3% (w/w), less than 0.2% (w/w), less than 0.1% (w/w), less than 0.08% (w/w), less than 0.07% (w/w), less than 0.06% (w/w), less than 0.05% (w/w), less than 0.04% (w/w), less than 0.03% (w/w), less than 0.02% (w/w), or less than 0.01% (w/w) of a foam adjuvant based on the total weight of the formulation.

In the context of the present specification, the term “foam adjuvant” relates to compounds capable of increasing the foaming capacity of a formulation and/or stabilizing a foam. In particular, the term “foam adjuvant” relates to fatty acids and fatty alcohols having at least 6 carbon atoms. In particular, the formulation of the present invention may comprise or consist of:
(a) a nanoemulsion comprising
   (i) an aqueous component, present in an amount of 70% to 95% w/w, based on the total weight of the nanoemulsion (a).
   (ii) nanovesicles, comprising
      (1) 1-5% of at least one phospholipid, based on the total weight of the nanoemulsion (a);
      (2) 2-10% of at least one polyoxyethylene-type surfactant, based on the total weight of the nanoemulsion (a);
      (3) 1-5% C₃ to C₅ alcohol, based on the total weight of the nanoemulsion (a); and
      (4) 2-10% triglycerides, based on the total weight of the nanoemulsion (a);
(b) 0.01-1% of a highly lipophilic macrolide lactone, based on the total weight of the formulation; and
(c) 0.1-10%, preferably 0.1-5%, more preferably 0.1-2% of at least one preservative, based on the total weight of the formulation;
(d) optionally, 0.1-30%, preferably 0.1-20%, more preferably 0.1-10% of at least one gelling agent, based on the total weight of the formulation;
wherein the formulation preferably has a pH of 2-7. Preferably, the formulation can comprise two surfactants, more preferably soy lecithin and Polysorbate 80.

In this formulation, the at least one phospholipid, the at least one polyoxyethylene-type surfactant, the C₃ to C₅ alcohol, the triglycerides, and the at least one preservative may be independently selected according to the herein-described disclosure.

In particular, the formulation of the present invention may comprise, essentially consist of or consist of:
(a) an emulsion of nanovesicles comprising
   (i) an aqueous component, present in an amount of 70% to 95% w/w, based on the total weight of the nanoemulsion (a).
   (ii) nanovesicles, comprising
      (1) 1-5% of at least one phospholipid, based on the total weight of the nanoemulsion (a);
      (2) 2-10% of at least one polyoxyethylene-type surfactant, based on the total weight of the nanoemulsion (a);
      (3) 1-5% C₃ to C₅ alcohol, based on the total weight of the nanoemulsion (a); and
      (4) 2-10% triglycerides, based on the total weight of the nanoemulsion (a);
(b) 0.01-1% of a highly lipophilic macrolide lactone, preferably tacrolimus, based on the total weight of the formulation;
(c) 0.1-10%, preferably 0.1-5%, more preferably 0.1-2% of at least one preservative, based on the total weight of the formulation;
(d) optionally, 0.1-30%, preferably 0.1-20%, more preferably 0.1-10% of at least one gelling agent, based on the total weight of the formulation;
wherein the formulation preferably has a pH of 2-7.

Preferably, the formulation can comprise two surfactants, more preferably soy lecithin and Polysorbate 80.

In this formulation, the at least one phospholipid, the at least one polyoxyethylene-type surfactant, the C₃ to C₅ alcohol, the triglycerides, and the at least one preservative may be independently selected according to the herein-described disclosure. This formulation may comprise further components selected from EDTA, α-tocopheryl acetate and citric acid. These components can be part of the aqueous component and/or of the nanovesicles.

In preferred embodiments, the formulation of the present invention may comprise or consist of:
(a) a nanoemulsion comprising
   (i) an aqueous component, present in an amount of 70-95% w/w, based on the total weight of the nanoemulsion (a).
   (ii) nanovesicles, comprising
      (1) 1-5% of at least one phospholipid, based on the total weight of the nanoemulsion (a);
      (2) 2-10% of at least one polyoxyethylene-type surfactant, based on the total weight of the nanoemulsion (a);
      (3) 1-5% C₃ to C₅ alcohol, based on the total weight of the nanoemulsion (a); and
      (4) 2-10% triglycerides, based on the total weight of the nanoemulsion (a);
(b) 0.01-1% pharmaceutically acceptable highly lipophilic macrolide lactone, preferably a calcineurin inhibitor, such as tacrolimus, based on the total weight of the formulation;
(c) optionally 0.5-6% of at least one gelling agent, based on the total weight of the formulation;
(d) optionally 0.1-2% of at least one preservative, based on the total weight of the formulation; and
(e) a propellant,
wherein the formulation is comprised in a pressurized container and wherein the propellant is provided to pressurize the container to provide a pressurized formulation.

The present invention also relates to a nanovesicle, comprising, essentially consisting of or consisting of
(i) 16-19% w/w of soy lecithin,
(ii) 32-36% w/w of Polysorbate 80,
(iii) 32-36% w/w of caprylic/capric triglycerides, and
(iv) 12-16% w/w of isopropyl alcohol.

A preferred nanovesicle of the invention comprises essentially consists of or consists of
(i) 17% w/w of soy lecithin
(ii) 34% w/w of Polysorbate 80
(iii) 35% w/w of caprylic/capric triglycerides
(iv) 14% w/w of isopropyl alcohol.

The present invention also relates to a nanoemulsion, comprising, essentially consisting of or consisting of
(a) 1.6-3.6% w/w of soy lecithin
(b) 3.3-6.9% w/w of Polysorbate 80
(c) 3.3-7.0% w/w of caprylic/capric triglycerides
(d) 1.3-2.9% w/w of isopropyl alcohol
(e) aqueous phosphate buffer, for example aqueous 5-100 mM phosphate buffer, pH 2-8, preferably pH 2-7, more preferably pH 2-5, ad 100%.

A preferred nanoemulsion of the present invention comprises, essentially consists of or consists of:
(a) 1.7% w/w of soy lecithin
(b) 3.4% w/w of Polysorbate 80
(c) 3.5% w/w of caprylic/capric triglycerides
(d) 1.4% w/w of isopropyl alcohol
(e) aqueous phosphate buffer (5 mM to 100 mM, preferably 10 mM to 50 mM), pH 6, ad 100%.

This nanoemulsion is termed herein “BF200”. The BF200 nanoemulsion can be obtained by contacting a mixture of ingredients (a)-(d) in a total amount of 10% w/w and 90% w/w of an aqueous 10 mM phosphate buffer, pH 6, under condition allowing formation of a nanoemulsion, thereby forming the nanoemulsion. An exemplary method for manufacture of the BF200 formulation is described in Example 1.

Another preferred nanoemulsion of the present invention comprises, essentially consists of or consists of:
(a) 2-3% w/w of soy lecithin
(b) 4.5-5.5% w/w of Polysorbate 80
(c) 4.5-5.5% w/w of caprylic/capric triglycerides
(d) 2-3% w/w of isopropyl alcohol
(e) aqueous 10 mM phosphate buffer, pH 6 or less, ad 100%.

This nanoemulsion is termed herein “BF215”. The BF215 nanoemulsion can be obtained by contacting a mixture of ingredients (a)-(d) in a total amount of 15% w/w and 85% w/w of an aqueous 10 mM phosphate buffer, pH 6, under condition allowing formation of a nanoemulsion, thereby forming the nanoemulsion. An exemplary method for manufacture of the BF215 formulation is described in Example 1.

Yet another preferred nanoemulsion of the present invention comprises, essentially consists of or consists of:
(a) 3-4% w/w of soy lecithin
(b) 6-7% w/w of Polysorbate 80
(c) 6-8% w/w of caprylic/capric triglycerides
(d) 2-4% w/w of isopropyl alcohol
(e) aqueous 10 mM phosphate buffer, pH 6, ad 100%.

This nanoemulsion is termed herein “BF220”. The BF220 nanoemulsion can be obtained by contacting a mixture of ingredients (a)-(d) in a total amount of 20% w/w and 80% w/w of an aqueous 10 mM phosphate buffer, pH 6, under condition allowing formation of a nanoemulsion, thereby forming the nanoemulsion. An exemplary method for manufacture of the BF220 formulation is described in Example 1.

All definitions and embodiments described for the first aspect are also envisioned for all other aspects described herein, where applicable.

Yet another aspect of the present invention relates to the formulations, as described herein, for use in medicine.

Yet another aspect of the present invention relates to the formulations as described herein for use in medicine.

Yet another aspect of the present invention relates to the formulations as described herein, for use in a method of treatment and/or prevention of a dermatological, ophthalmic or autoimmune disease or condition, or for the prevention of organ rejection after transplantation.

The dermatological disease or condition to be treated with the formulation as described herein, may include, but is not limited to, diseases or conditions of the skin, skin appendages or mucosa.

The dermatological disease or condition to be treated with the formulation as described herein, may be selected from the group consisting of inflammatory, neoplastic, proliferative, infectious, and/or autoimmune diseases or conditions, and/or the cutaneous manifestation thereof, and/or diseases associated with single lesions or fields of lesions, neoplastic, proliferative and/or inflammatory changes.

The inflammatory dermatological disease or condition to be treated with the formulation as described herein, may be selected from the group consisting of dermatitis, contact dermatitis, acne, atopic dermatitis, eczema, pustular dermatitis, seborrheic dermatitis, perioral dermatitis, chronic wound, urticaria, skin ulcer, rosacea, rash, drug eruptions, toxic epidermal necrolysis;

erythema multiforme, erythema nodosum, granuloma annulare, and other cutaneous manifestations of inflammation.

The dermatological disease or condition may be an autoimmune dermatological disease or condition. The autoimmune dermatological disease or condition, or the cutaneous manifestation of the autoimmune condition to be treated with the formulation as described herein, may be selected from the group consisting of psoriasis, pemphigus, systemic lupus erythematodes, lichen planus, morphea, sclerodermia, epidermolysis bullosa, dermatomyositis, graft-versus-host syndrome.

Further diseases or conditions to be treated with the formulation as described herein, may be selected from the group consisting of organ rejections after organ transplants (such as heart, kidney, liver, lung transplants).

The ophthalmic disease or condition to be treated with the formulation as described herein, may be selected from the group consisting of keratoconjunctivitis (AKC), vernal keratoconjunctivitis (VKC), dry-eye, corneal endothelial rejection after corneal transplantation.

Yet another aspect of the present invention is a method for the preparation of the formulation as described herein, comprising the following steps:
(a) mixing the at least one lipophilic component, the at least one surfactant, and the at least one alcohol having at least three carbon atoms,
(b) mixing an active agent into the lipid phase and
(c) contacting the mixture obtained in step (a) with an aqueous component, under conditions allowing formation of a nanoemulsion.

In step (b), the conditions allowing formation of the nanoemulsion may include mixing both phases at an appropriate temperature and stirring in a fashion to form nanovesicle. The skilled person knows suitable temperature and stirring conditions. A vesicle size of less than or equal to 500 nm or less than or equal to 300 nm, preferably in the range of 5 nm to 200 nm, more preferably in the range of 5 nm to 100 nm can be obtained. In particular, the nanoemulsion of the present invention according to step (b) can be prepared without the use of high energy methods, which are well known in the art. High energy method includes high-pressure homogenization, microfluidization, and ultrasonication (Prev Nutr Food Sci. 2019 September; 24 (3): 225-234).

The method comprises a step of adding a lipophilic active agent to the lipid phase until complete solution. Preferably, the active agent is tacrolimus.

The method for the preparation the pharmaceutical formulation of the present invention can further comprise:
(i) adding a gelling agent, and/or
(ii) adding a preservative.

Yet another aspect of the present invention is a dispenser product or container product, comprising the formulation as described herein. In the dispenser product or container product, the formulation of the present invention is provided in a container or dispenser. Suitable dispensers and containers are known to the skilled person. For example, the dispenser may be a squeeze tube, comprising the formulation as described herein. The squeeze tube may contain the gel formulation as described herein. The dispenser may also be a metered dose dispenser or a foam dispenser or a spray dispenser.

The container or foam dispenser or spray dispenser may comprise a propellant, wherein the propellant is provided to pressurize the container or foam dispenser or spray dispenser. Any suitable propellant may be used. Suitable propellants and mixtures thereof are known to the skilled person. Preferably, the propellant is selected from propane, isobutane, n-butane and mixtures thereof.

The dispenser product may be a foam dispenser or spray dispenser product, comprising a foam dispenser or spray dispenser, as described herein, the foam dispenser or spray dispenser comprises a container, wherein said container comprises the formulation as described herein, and a propellant. The propellant is provided to pressurize the foam dispenser or spray dispenser. Any suitable propellant may be used, as described herein. A foam-generating device or spray-generating device is mounted on the container. In particular, the formulation is prepared as a foamable formulation.

Yet another aspect of the present invention is the use of the formulation of the present invention, as described herein, for the manufacture of a medicament for topical or systemic treatment and/or prevention of a dermatological, ophthalmic, or autoimmune disease or condition in a subject or for prevention of organ rejection after transplantation.

Yet another aspect of the present invention is a method of treatment and/or prevention of a dermatological disease or condition in a subject, said method comprising administering to the subject, a pharmaceutically effective amount of the formulation as described herein. In particular, the dermatological disease is a dermatological disease or condition as described herein.

The invention also pertains to the following items:
1. A formulation comprising
   (a) a nanoemulsion, said emulsion comprising:
      (i) at least one aqueous component;
      (ii) a carrier component comprising:
         (1) at least one lipophilic component,
         (2) at least one surfactant, and
         (3) at least one alcohol; and
   (b) an active agent, wherein the active agent is a highly lipophilic macrolide lactone, preferably tacrolimus.
5. The formulation of any of the preceding items, wherein the at least one alcohol comprises at least three carbon atoms, preferably 3, 4 or 5 carbon atoms.
7. The formulation of any of the preceding items, wherein the active agent is present in an amount of from 0.001% to 5% (w/w), preferably 0.005% to 1% (w/w), more preferably 0.01% to 1.0% (w/w), based on the total weight of the formulation.
12. The formulation of any of the preceding items, wherein formulation is a pharmaceutical formulation.
13. The formulation of any of the preceding items, wherein formulation is for topical or oral use.
14. The formulation of any of the preceding items, wherein the active agent is dissolved in the carrier component.
15. The formulation of any of the preceding items, wherein the active agent has a logP value of 3 or higher.
16. The formulation of any one of the preceding items, wherein the nanoemulsion comprises nanovesicles, wherein the nanovesicles have a size of less than or equal to 500 nm, preferably in the range of 5 nm to 200 nm, preferably 5 nm to 100 nm when stored for one month, two months or three months at 40° C.
17. The formulation of any one of the preceding items, wherein the nanoemulsion comprises nanovesicles, wherein the nanovesicles have a size of less than or equal to 500 nm, preferably in the range of 5 nm to 200 nm, more preferably 5 nm to 100 nm when stored for six months, twelve months, eighteen months or 24 months at 25° C.
18. The formulation of any one of the preceding items, wherein the aqueous component is present in an amount of 50% to 99% w/w, based on the total weight of the nanoemulsion (a), preferably from 70% to 95% (w/w), and more preferably from 80% to 95% (w/w).
19. The formulation of any one of the preceding items, wherein the aqueous component comprises at least one pH buffering agent.
20. The formulation of item 19, wherein the pH buffering agent is selected from the group consisting of citrate, phosphate, acetate and carbonate.
21. The topical or oral formulation of any one of the preceding items, having a pH of 2-7.
22. The parenteral or ophthalmic formulation of any one of the preceding items, having a pH of 5-9.
23. The formulation of any one of the preceding items, wherein the at least one lipophilic component is selected from triglycerides and mixtures thereof.
24. The formulation of any one of the preceding items, wherein the at least one lipophilic component comprises a caprylic and/or a capric triglyceride or a mixture thereof.
25. The formulation of any one of the preceding items, wherein the at least one lipophilic component is present in an amount of from 0.1% to 30% (w/w), based on the total weight of the nanoemulsion (a), preferably from 0.25% to 10% (w/w), and more preferably from 0.5% to 8% (w/w) or 3% to 8% (w/w).
26. The formulation of any one of the preceding items, wherein the at least one surfactant comprises (a) a phospholipid, a lysophospholipid, a ceramide and/or a mixture thereof, and/or (b) a polyoxyethylene-type surfactant.
27. The formulation of any one of the preceding items, wherein the at least one surfactant comprises lecithin, preferably soy lecithin.
28. The nanoemulsion of item 27, wherein the lecithin has a phosphatidylcholine content of at least 80% by weight.
29. The formulation of any one of items 26-28, wherein the phospholipid, the lysophospholipid, the ceramide and/or the mixture thereof is present in an amount of from 0.1% to 10% (w/w), based on the total weight of nanoemulsion (a), preferably from 0.15% to 5% (w/w), and more preferably from 0.2% to 3% (w/w).
30. The formulation of any one of the items 26-29, wherein the polyoxyethylene-type surfactant comprises Polysorbate 80.
31. The formulation of any one of items 26-30, wherein the polyoxyethylene-type surfactant is present in an amount of from 0.1% to 10% (w/w), based on the total weight of the nanoemulsion (a), more preferably from 0.2% to 5% (w/w), and most preferably from 0.5% to 5% (w/w).
32. The formulation of any one of the preceding items, wherein at least one alcohol is selected from the group consisting of 1-propanol or 2-propanol and a mixture thereof.
33. The formulation of any one of the preceding items, wherein the at least one alcohol is present in an amount of from 0.1% to 10% (w/w), based on the total weight of the nanoemulsion (a), preferably from 0.5% to 5% (w/w), and more preferably from 1% to 2% (w/w).
34. The formulation of any one of the preceding items, comprising at least one gelling agent.
35. The formulation of any one of the preceding items, wherein the gelling agent is selected from the group consisting of poloxamer, xanthan gum, bentonite, sodium carboxymethylcellulose, hydroxymethyl cellulose, carbomer, hydroxypropyl cellulose, gellan gum, guar gum, pectin, poly(ethylene) oxide, polycarbophil, alginate, tragacanth, povidone, gelatin, and mixtures thereof.
36. The formulation of item 34 or 35, wherein the gelling agent is selected from poloxamer, xanthan and/or mixtures thereof.
37. The formulation of any one of the preceding items, wherein the gelling agent is present in an amount of from 0.1% to 10% (w/w), based on the total weight of the formulation, preferably from 0.25% to 5% (w/w), and more preferably from 1% to 4% (w/w).
38. The formulation of any one of the preceding items, further comprising at least one preservative.
39. The formulation of item 38, wherein the preservative is benzoate, preferably sodium benzoate.
40. The formulation of item 38 or 39, wherein the preservative is present in an amount of from 0.01% to 3% w/w, based on the total weight of the formulation, preferably from 0.2% to 2% (w/w), and more preferably from 0.2% to 1.5% (w/w).
41. The formulation of any one of the preceding items, which is essentially free of parabens.
42. The formulation of any one of the preceding items, characterized by a polydispersity index of less than or equal to 0.8, wherein the polydispersity index is determined by dynamic light scattering.
43. The formulation of any one of the items 1-42, comprising:
   (a) an emulsion of nanovesicles comprising
      (i) an aqueous component, present in an amount of 70% to 95% w/w, based on the total weight of the nanoemulsion (a).
      (ii) nanovesicles, comprising
         (1) 1-5% of at least one phospholipid, based on the total weight of the nanoemulsion (a);
         (2) 2-10% of at least one polyoxyethylene-type surfactant, based on the total weight of the nanoemulsion (a);
         (3) 1-5% C₃ to C₅ alcohol, based on the total weight of the nanoemulsion (a); and
         (4) 2-10% triglycerides, based on the total weight of the nanoemulsion (a);
   (b) 0.01-1% of a highly lipophilic macrolide lactone or a combination thereof, based on the total weight of the formulation;
   (c) 0.1-10% of at least one preservative, based on the total weight of the formulation and wherein the formulation preferably has a pH of 2-7.
44. The formulation of any one of the preceding items, provided in a container, wherein the container further comprises a propellant, and wherein the propellant is provided to pressurize the container and/or to pressurize the formulation in the container.
45. The formulation of any one of the preceding items, for use in medicine.
46. The formulation of any one of the preceding items, for use in a method of topical or systemic treatment and/or prevention of a dermatological, ophthalmic or autoimmune disease or condition in a subject or for the prevention of organ rejection after transplantation.
47. The formulation for use of item 46, wherein the ophthalmic disease is selected from the group consisting of keratoconjunctivitis (AKC), vernal keratoconjunctivitis (VKC), dry-eye, corneal endothelial rejection after corneal transplantation.
48. The formulation for use of item 46, wherein the dermatological disease or condition includes diseases or conditions of the skin, skin appendages or mucosa.
49. The formulation for use of any one of the items 46 or 48, wherein the dermatological disease or condition is selected from the group consisting of inflammatory, neoplastic, proliferative, infectious, and/or autoimmune diseases or conditions, and/or the cutaneous manifestation thereof, and/or diseases associated with single lesions or fields of lesions, neoplastic, proliferative and/or inflammatory changes.
50. The formulation for use of item 49, wherein the inflammatory dermatological disease or condition is selected from the group consisting of dermatitis, contact dermatitis, acne, atopic dermatitis, eczema, pustular dermatitis, seborrheic dermatitis, perioral dermatitis, chronic wound, urticaria, skin ulcer, rosacea, rash, drug eruptions, toxic epidermal necrolysis; erythema multiforme, erythema nodosum, granuloma annulare, and other cutaneous manifestations of inflammation.
51. The formulation for use of item 49, wherein the autoimmune dermatological disease or condition, or the cutaneous manifestation of the autoimmune condition is selected from the group consisting of psoriasis, pemphigus, systemic lupus erythematodes, lichen planus, morphea, sclerodermia, epidermolysis bullosa, dermatomyositis, graft-versus-host syndrome.
52. A dispenser product, comprising the formulation of any one of the items 1-51.
53. A container, comprising the formulation of any one of the items 1-51.
54. Use of the formulation according to any one of the items 1-51, for the manufacture of a medicament for the treatment and/or prevention of a dermatological disease or condition in a subject.
55. A method of treatment and/or prevention of a dermatological disease or condition in a subject, said method comprising administering to the subject, a pharmaceutically effective amount of the formulation of any one of the items 1 to 57.

The present invention is further illustrated by the following figures and Examples.

### FIGURE LEGENDS

FIG. 1: Solubility of tacrolimus. Top: Solubility tests with tacrolimus (time point 0). From left to right: TC2 (1 mg TC/ml aqueous phosphate buffer), TC3 (20 mg TC/g lipid phase or carrier component), TC4 (2 mg TC/ml BF200 nanoemulsion), TC5 (1 mg TC/ml BF200 nanoemulsion); Bottom: Solubility tests with tacrolimus after 6 months storage at 5° C. and 25° C. From left to right: TC4 stored at 5° C., TC4 stored at 25° C., TC5 stored at 5° C., TC5 stored at 25° C.

FIG. 2: Tacrolimus assay in nanoemulsion formulations with different nominal TC content (0.1%, 0.01%) after storage at 2-8° C., 25° C. or 40° C.

FIG. 3: Particle size in nanoemulsion formulations with different nominal TC content (0.1%, 0.01%) after storage at 2-8° C., 25° C. or 40° C.

FIG. 4: Polydispersity index (PDI) of formulations with different nominal TC content (0.1%, 0.01%) after storage at 2-8° C., 25° C. or 40° C.

FIG. 5: In Vitro Release (SUPAC-SS) after 2.6 hours of tacrolimus formulations with nanoemulsion compared to commercially available tacrolimus ointments.

FIG. 6: Epidermal penetration of tacrolimus formulation with nanoemulsion compared to commercially available tacrolimus ointment.

Some data shown in the figures result from pooled experiments.

### EXAMPLES

### Example 1: Preparation of Nanoemulsions BF200, BF215 and BF220

TABLE 1
Lipophilic content of nanoemulsions used in the Examples

| | |
|---|---|
| BF200 | 10% lipophilic content |
| BF215 | 15% lipophilic content |
| BF220 | 20% lipophilic content |

The qualitative and quantitative compositions of the nanoemulsions BF200, BF215 and BF220 are given in Table 2.

TABLE 2
Composition of nanoemulsions BF200, BF215 and BF220

| | | Content | Content | Content | | |
|---|---|---|---|---|---|---|
| | | (% w/w) | (% w/w) | (% w/w) | | |
| | Ingredient | BF200 | BF215 | BF220 | Function | Quality |
| 1 | Soy lecithin | 1.7 | 2.0-3.0 | 3.0-4.0 | surfactant | >90% |
| | | | | | | phosphadidylcholine, |
| | | | | | | for pharmaceutical |
| | | | | | | use, USP |
| 2 | Polysorbate 80 | 3.4 | 4.5-5.5 | 6.0-7.0 | surfactant | Ph. Eur. |
| | (polyoxyethylene | | | | | |
| | sorbitol monooleate) | | | | | |
| 3 | Caprylic/capric | 3.5 | 4.5-5.5 | 6.0-8.0 | lipid core | Ph. Eur. |
| | triglycerides | | | | | |
| 4 | Isopropyl alcohol | 1.4 | 2.0-3.0 | 2.0-4.0 | solvent | Ph. Eur. |
| | Content (% w/w) | 10.0 | 15.0 | 20.0 | | |
| | ingredients 1-4 | | | | | |
| | Phosphate buffer | ad 100.00 | ad 100.00 | ad 100.00 | solvent | Water: Ph. Eur.; |
| | | | | | | Disodium phosphate |
| | | | | | | and sodium |
| | | | | | | hydrogen phosphate: |
| | | | | | | Ph. Eur. |

The manufacturing process for the nanoemulsions in a typical batch size consists of the following steps 1-4:

### Step 1: Preparation of Phosphate Buffer (Aqueous Component)

Phosphate buffer (1000 g), was prepared and the phosphate buffer optionally sterilized.

### Step 2: Preparation of the Carrier Component (Lipid Phase) Containing the Lipophilic Component, the Surfactants and the Alcohol

TABLE 3
Carrier component

| Ingredient | Weight (g) |
|---|---|
| Soy lecithin | 17 |
| Polysorbate 80 | 34 |
| (polyoxyethylene | |
| sorbitol | |
| monooleate) | |
| Caprylic/capric | 35 |
| triglycerides | |
| Isopropyl alcohol | 14 |

Soy lecithin (17 g) was weighed in a suitable vessel, isopropyl alcohol (14 g) was added and the vessel was covered to avoid alcohol evaporation. Soy lecithin was dissolved under continuous stirring with a suitable stirrer at room temperature. Caprylic/capric triglycerides (35b g) and Polysorbate 80 (34 g) were weighed and added to the solution of soy lecithin. The mixture was stirred with a suitable stirrer at room temperature until a homogenous clear solution was obtained. This solution is the carrier phase to be included in the nanoemulsion containing all emulsifiers and lipid components of the nanoemulsion BF200. According to this procedure BF215 and BF220 was prepared by adapting the amount of the components (see Table 2).

In all examples shown herein, nanoemulsion BF200 was used.

Step 3: Manufacturing of the Nanoemulsion by Mixing the Aqueous Component from Step 1 and the Carrier from Step 2 for a Lipid Content of 10% (BF200)

Manufacturing of an emulsion by mixing 900 g phosphate buffer (from Step 1) and 100 g carrier (from Step 2). First, the aqueous component comprising the phosphate buffer was heated to approximately 45-60° C. in a suitable vessel. Then, the carrier (concentrate) of step 2 was heated to approximately 45-60° C. Subsequently, the carrier was poured to the phosphate buffer under continuous stirring with a propeller mixer resulting in the formation of a stable trombe (or spout) having the maximal possible diameter without causing foaming or sputtering. The resulting nanoemulsion is stirred for 15 min. Finally, the nanoemulsion was cooled down to room temperature.

In nanoemulsion BF215, 850 g phosphate buffer (from Step 1) and 150 g carrier were mixed. In nanoemulsion BF220, 800 g phosphate buffer (from Step 1) and 200 g carrier were mixed.

### Step 4: Preparation of the Final Formulation and Primary Packaging

Optionally, the nanoemulsion can be sterilized.

Depending on the purpose of the nanoemulsion, one may add adjuvants and/or excipients and/or active ingredients (at the appropriate step according to the description) and/or dilute the nanoemulsion with in a way to get a suitable pharmaceutical formulation, e.g., by adding water, a suitable buffer, or an additional aqueous gel base with e.g., poloxamer 407 or xanthan gum.

### Example 2: Preparation of a Nanoemulsion Formulation (BF220) Containing 0.1% or 0.01% Tacrolimus (TC) and 4% Poloxamer 407

The formulations were prepared according to Example 1 with the addition of the appropriate amount of an aqueous gel base with poloxamer 407 in step 4. TC in an amount of 0.1% or 0.01% was added to the lipophilic component of step 2 from example 1.

### Example 3: Determination of the Solubility Properties of Tacrolimus in the Nanoemulsion Formulation

Tacrolimus was dissolved by mechanical techniques (stirring). Determination of a complete solution of substance was based on visual observation. A complete dissolution was defined as a clear solution with no signs of cloudiness or precipitation. The pure solvents were used as reference.

### Example 4: Determination of Vesicle Size and Polydispersity Index by Dynamic Light Scattering

The size of the nanovesicles, expressed as the z-average size (e.g., in nm), and the homogeneity of nanovesicle formulations, expressed as polydispersity index was determined by dynamic light scattering (sometimes referred as Photon Correlation Spectroscopy (PCS) or Quasi-Elastic Light Scattering (QELS)). The technique is well known in the art and well established to determine size of nano or micro particles or vesicles in emulsions, suspensions or polymeric solutions with a laser. Measurements were conducted with an Zetasizer Nano ZS (Malvern Instruments Ltd, Malvern, Worcestershire, UK). The measurement was performed according to the manufacturer's instructions.

The Zetasizer Nano ZS is instrumented with a 633 nm green laser and optics with a 173°scattering detector angle for size measurement. The device may be operated under vacuum for measurements, but in these cases, vacuum was not applied to the samples for size and homogeneity measurements.

### Example 5: In Vitro Release (SUPAC-SS) of Tacrolimus Formulations with Nanoemulsion and Tacrolimus Ointments

In vitro release method is based on an open chamber diffusion cell system such as a Franz cell system. The cylindrical glass Franz cell is a diffusion chamber comprising an upper and a lower part between which the synthetic membrane (e.g., EMD Millipore MF Membrane; 0.025 μm) is clamped. The lower part (approx. 7 mL) is filled with an acceptor medium (EtOH/H₂O 75:25 v/v %) maintained at a temperature of about 32° C. in which the API has a sufficient solubility. The acceptor medium is stirred (approx. 400 rpm) to ensure the partition and dissolution of the API. The diffusion area of the membrane is approximately 1.8 cm² (25 mm diameter). Diffusion of tacrolimus from a topical product to and across the membrane was monitored (up to 2.6 hours) by assay using high-performance liquid chromatography (HPLC-UV).

### Example 6: Epidermal Penetration of Tacrolimus Formulation with Nanoemulsion Compared to Commercially Available Tacrolimus Ointment

The nanoemulsion formulation with 0.1% tacrolimus was compared to the conventional 0.1% ointment in a skin penetration study by assessing tacrolimus deposition in a layer wise assay using ex-vivo facial skin. An established ex-vivo model was used to explore drug penetration into human skin from routine facial aesthetic surgery. The penetration test is dived into the following steps:
I. Skin sampling: Samples from blepharoplasty (upper and lower eyelids) and rhytidectomy (cheek/periauricular) were taken.
II. Sample preparation:
   a. One non-occlusive dermal application to the ex vivo skin samples of 6 mm diameter.
   b. Approx. 28.3 mg (100 mg/cm²) of test items
   c. After incubation at approx. 37° C. and 5% CO₂ in humidified atmosphere the test formulation was washed of samples with 70% ethanol. The samples were frozen in freezing medium at −80° C.
   d. Cryosectioning at −28° C. with a thickness of 10 μm
   e. Extraction of tacrolimus with ACN at about 20° C. for 24 hours and 450 rpm followed by centrifugation at 20° C.
III. Tacrolimus content by assay using high-performance liquid chromatography (HPLC-UV).

### Example 7: Determination of Viscosity

Viscosity was measured by rotation (measuring geometry: cone/plate) with a constant shear rate of 90.0 s⁻¹ at 20° C.

### Example A

Tacrolimus (TC) Content Over Time in BF220 with PX=4% as Gelling Agent at Different Temperatures

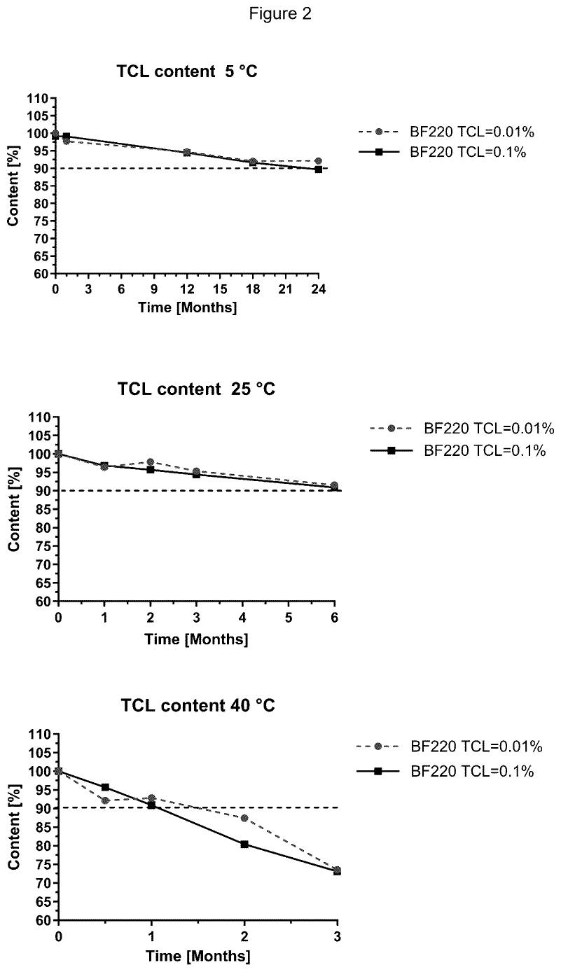

Nanoemulsion formulations BF220 TC=0.1% and BF220 TC=0.01% were prepared as described in Example 2. The nanoemulsion formulations were stored at 2-8° C., 25° C. and 40° C. The content of TC was determined at 0 (starting point) and various time points during the 24 months storage period. The results are shown in FIG. 2.

Conclusion: At 2-8° C.: Formulation (Example 2) preserve the API (TC) content better than conventional aqueous formulations. TC in formulation of Example 2 was found to be stable after 24 months storage at 2-8° C. At 40° C. TC in formulation of Example 2 is stable for at least 1 month.

### Example B

Particle Size and Particle Size Distribution Over Time in BF220 with PX=4% as Gelling Agent at Different Temperatures

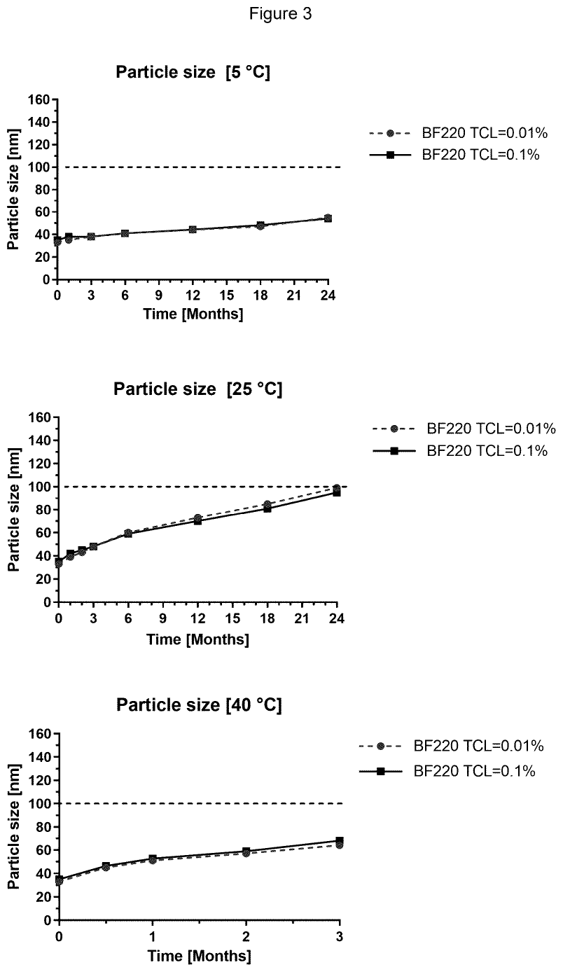

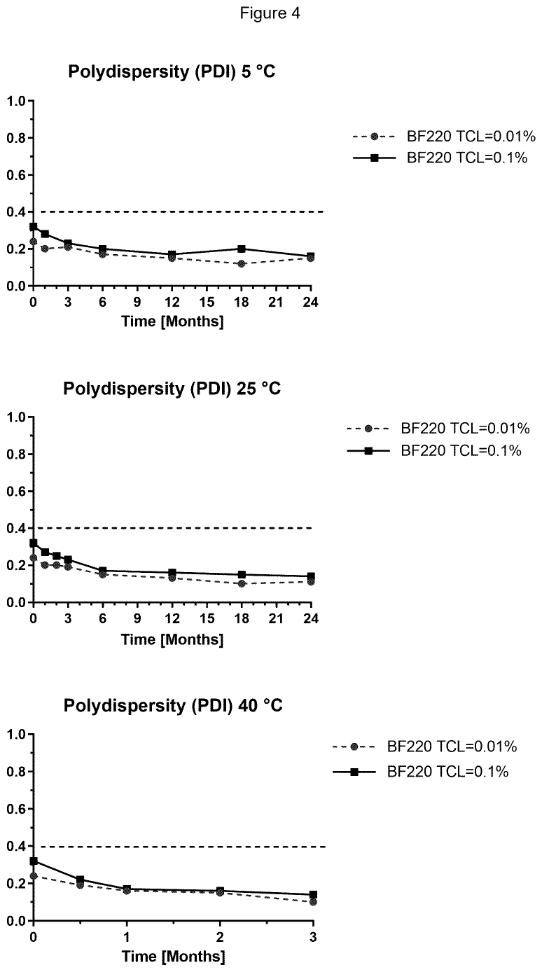

Nanoemulsion formulations BF220 TC=0.1% and BF220 TC=0.01% were prepared as described in Example 2. The nanoemulsion formulations were stored at 2-8° C., 25° C. and 40° C. The particle size, particle size distribution and pH were determined at 0 (starting point) and various time points during the 24 months storage period. The results are shown in FIG. 3 and FIG. 4.

Conclusion: At 2-25° C.: Formulations (Example 2) containing API (TC) were found to be stable for at least 24 months storage at 2-25° C. At 40° C. the formulations are stable for at least 3 months.

### Example C

In Vitro Release (SUPAC-SS) of Tacrolimus Formulations with Nanoemulsion and Tacrolimus Ointments

From both test formulations a significant higher amount of tacrolimus is released and can permeate into and through the skin if the formulations are applied. The properties of the nanoemulsion formulation contribute to a more effective release of tacrolimus than from an ointment, where tacrolimus diffusion is further inhibited by the ointment.

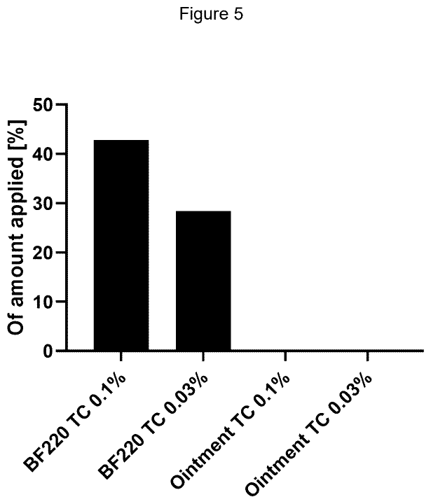

Using the method for release testing for tacrolimus nanoemulsion, no tacrolimus has been released from the reference ointments with a tacrolimus content of 0.1% and 0.03%. From the test formulation with nanoemulsion BF220 and 0.03% tacrolimus, 28.39% of the applied amount tacrolimus were released. From the test formulation with nanoemulsion BF220 and 0.1% tacrolimus, 42.81% of the applied amount tacrolimus were released. The data is presented in FIG. 5.

### Example D

### Solubility of Tacrolimus in Nanoemulsion Formulations

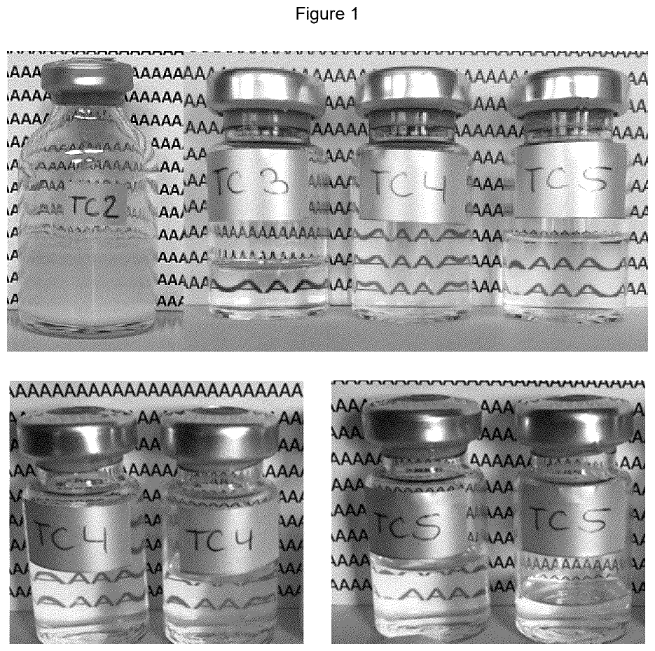

Samples were prepared as explained in Example D. The compound was dissolved in 10 mM Phosphate buffer (TC2) at defined concentration (see table below). For samples TC3, TC4 and TC5 the lipid phase (intermediate) of nanoemulsion BF200 was used as solvent. In a first step, Tacrolimus was dissolved in the lipid phase, followed by the preparation of the nanoemulsion by combining the aqueous phase with the lipid phase (containing Tacrolimus). Samples TC4 and TC5 were stored at 5° C. and 25° C. for a period of 6 months. The results of solubility tests for time point 0 and 6 months are shown in FIG. 1 and Table 4.

TABLE 4
Results of solubility tests

| Tacrolimus | | | Name of test | Solution | Solution | |
|---|---|---|---|---|---|---|
| concentration | | Solvent | solution | Appearance (T0) | Appearance (T6) | Solubility |
| 1 | mg/mL | Phosphate Buffer | TC2 | Turbid, white | Turbid, white | Insoluble |
| | | | | precipitate | precipitate | |
| 20 | mg/g | Lipid phase of | TC3 | Clear, yellow color | Clear, yellow | Soluble |
| | | nanoemulsion | | | color | |
| | | BF200 | | | | |
| 2 | mg/mL | 100% nanoemulsion | TC4 | Clear, slightly | Clear, slightly | Soluble |
| | | BF200 | | opalescent | opalescent | |
| 1 | mg/mL | 50% nanoemulsion | TC5 | Clear, slightly | Clear, slightly | Soluble |
| | | BF200 | | opalescent | opalescent | |

Conclusion: Tacrolimus is highly soluble in the aqueous nanoemulsion (at least 90% water content) in a concentration of at least 2 mg/mL.

### Example E

Epidermal Penetration of Tacrolimus Formulation with Nanoemulsion Compared to Commercially Available Tacrolimus Ointment

The results indicate that tacrolimus from the conventional lipophilic ointment containing 0.1% tacrolimus mainly stays on top of the skin, presumably associated to its formulation, and does not penetrate through the epidermis, while a higher tacrolimus deposition in the deeper skin layers can be achieved by the formulation with nanoemulsion (BF220) containing 0.1% tacrolimus. This outcome is in line with the results of Example C.

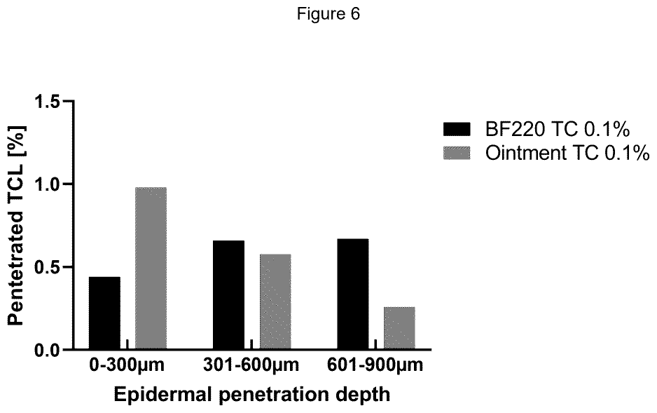

After 24 h of incubation, differences in the deposition profile of samples treated with nanoemulsion (BF220) containing 0.1% tacrolimus and conventional lipophilic ointment containing 0.1% were observed. It was found that the amount of TC after treatment with nanoemulsion (BF220) containing 0.1% tacrolimus was very evenly distributed throughout the three skin layers, with a tendency to rather accumulate towards the deeper dermis. During treatment with conventional lipophilic ointment containing 0.1%, the highest amount of tacrolimus is found in the upper layer of the skin, with the concentration steeply decreasing with the depth of the layer. The data is presented in FIG. 6.

1. A formulation comprising
(a) a nanoemulsion comprising:
(i) at least one aqueous component;
(ii) a carrier component comprising:
(1) at least one lipophilic component,
(2) at least one surfactant, and
(3) at least one alcohol; and
(b) an active agent, wherein the active agent is a highly lipophilic macrolide lactone.

2. The formulation of claim 1, wherein the macrolide lactone has a logP value of 3 or higher.

3. The formulation of claim 1, wherein the active agent is dissolved in the carrier component of the nanoemulsion.

4. The formulation of claim 1, wherein the active agent is Tacrolimus, Pimecrolimus, Everolimus or Sirolimus, preferably Tacrolimus, a derivative, an isomeric form, a tautomeric form, a precursor, a metabolite, hydrate, and/or a pharmaceutically acceptable salt thereof.

5. The formulation of claim 1, wherein the active agent is present in an amount from 0.001% to 25% (w/w), preferably 0.005% to 1% (w/w), most preferably 0.01% to 1% (w/w) based on the total weight of the formulation.

6. The formulation of claim 1, wherein the aqueous component is present in an amount from 70% to 95% (w/w), and more preferably from 80% to 95% (w/w) based on the total weight of the nanoemulsion.

7. The formulation of claim 1, comprising a total aqueous component in an amount of at least 45%, at least 50%, at least 55%, at least 60%, at least 65%, at least 70%, at least 75% or at least 80% (w/w), based on the total weight of the formulation, preferably from 50% to 99% (w/w), from 70% to 95% (w/w), more preferably from 75% to 95% (w/w), even more preferably from 80% to 90% (w/w).

8. The formulation of claim 1, wherein
(a) the at least one alcohol comprises at least three carbon atoms, preferably 3, 4 or 5 carbon atoms, preferably wherein the at least one alcohol is selected from the group consisting of 1-propanol or 2-propanol and mixtures thereof;
(b) the at least one lipophilic component is selected from triglycerides and mixtures thereof, preferably wherein the at least one lipophilic component is a caprylic and/or a capric triglyceride or a mixture thereof; and/or
(c) the at least one surfactant is selected from the group consisting of a phospholipid, a lysophospholipid, a ceramide and/or a mixture thereof, and/or the at least one surfactant is a polyoxyethylene-type surfactant.

9. The formulation of claim 1, wherein the at least one surfactant is phosphatidylcholine.

10. The formulation of claim 1, wherein the formulation comprises from 0.5% to 5% (w/w), preferably from 1% to 4% (w/w), more preferably from 1.2% to 3.5% (w/w) phosphatidylcholine.

11. The formulation of claim 1, wherein the formulation comprises a propellant and is comprised in a pressurized container.

12. The formulation of claim 1, wherein the formulation comprises
essentially no fatty alcohol; and/or
essentially no emollient selected from a monoester or diester comprising an alcohol and a fatty acid.

13. (canceled)

14. The formulation of claim 1, comprising at least one gelling agent.

15. The formulation of claim 1, wherein the active agent has a content of more than or equal to 80%, preferably more or equal to 85%, more preferably more or equal to 90% when
a) stored for one month, two months, three months, six months at 2-25° C.; or
b) stored for one month, two months, three months, six months, twelve months, eighteen months or twenty-four months at 2-8° C.

16. The formulation of claim 1, wherein the nanoemulsion comprises nanovesicles, wherein the nanovesicles have a size as determined by dynamic light scattering of less than or equal to 500 nm, preferably less than or equal to 200 nm, more preferably in the range of 5 nm to 100 nm when
a) stored for one month, two months, three months, six months, twelve months, eighteen months, or twenty-four months at 2-25° C.; or
b) stored for one month, two months, three months, six months, twelve months, eighteen months, twenty-four months, thirty months, or thirty-six months at 2-8° C.

17. The formulation of claim 1, characterized by a polydispersity index as determined by dynamic light scattering of less than or equal to 0.4 when
a) stored for one month, two months, three months, six months, twelve months, eighteen months, or twenty-four months at 2-25° C.
b) stored for one month, two months, three months, six months, twelve months, eighteen months, twenty-four months, thirty months, thirty-six months at 2-8° C.

18. The formulation of claim 1, wherein the formulation is a topical formulation, an ophthalmic formulation, a parenteral formulation or an oral formulation.

19. The formulation of claim 1, wherein the formulation is a lotion, a spray, a foam, an emulsion, a nanoemulsion, a gel or a cream.

20. (canceled)

21. A method of treating a dermatological, ophthalmic or autoimmune disease or condition, or preventing organ rejection after transplantation, comprising administration of an effective amount of the formulation of claim 1 to a subject in need thereof.

22. A container or a foam dispenser or spray dispenser product comprising a container, comprising a formulation, wherein said formulation comprises a nanoemulsion comprising:
a. at least one aqueous component;
b. a carrier component comprising:
(1) at least one lipophilic component,
(2) at least one surfactant, and
(3) at least one alcohol; and
c. an active agent, wherein the active agent is a highly lipophilic macrolide lactone;
wherein the container further comprises a propellant, wherein the propellant is provided to pressurize the container.
